(19) United States
(12) Patent Application Publication
(10) Pub. No.: US 2024/0120034 A1
(43) Pub. Date: Apr. 11, 2024

(54) SEQUENTIAL OPTIMIZATION PROCEDURE FOR SYSTEMS WITH DEGENERATION

(71) Applicants: BASF SE, Ludwigshafen am Rhein (DE); Fraunhofer-Gesellschaft Zur Förderung Der Angewandten Forschung E.V., München (DE)

(72) Inventors: David Hajnal, Ludwigshafen (DE); Michael Bortz, Kaiserslautern (DE); Philipp Suess, Kaiserslautern (DE); Karl-Heinz Kuefer, Kaiserslautern (DE)

(21) Appl. No.: 18/268,987

(22) PCT Filed: Dec. 17, 2021
(86) PCT No.: PCT/EP2021/086465

§ 371 Date: Jun. 22, 2023

(30) Foreign Application Priority Data
Dec. 23, 2020 (EP) 20216902.5

(51) Int. Cl. G16C 20/50; G06F 17/11

(52) U.S. Cl. CPC G16C 20/50; G06F 17/11

(57) ABSTRACT

The present invention generally relates to a system optimization procedure. The method includes
a) providing, via an input channel, a system model for modelling the chemical mixture, which associates a set of design parameters with a plurality of objective parameters that represent design characteristics of the chemical mixture, wherein the set of design parameters comprises a chemical mixture recipe having two or more ingredients, and the plurality of objective parameters comprises two or more physicochemical properties of the chemical mixture;
b) defining, via the input channel, a set of primary optimization objective parameters,
c) performing, by a processor, a multi-objective optimizing process on the system model by exploring a plurality of design configurations by assigning specified values to the set of design parameters; and
d) determining, by the processor, if the multi-objective optimizing process yields a degenerated multi-objective optimal design.

### FIELD OF THE INVENTION

The present invention generally relates to system optimization, and in particular to a computer-implemented method and a device for generating a recipe profile of a chemical mixture, to a method and device for monitoring production of a chemical mixture, to a method and device for validating production of a chemical mixture, a computer program element, and a computer readable medium.

### BACKGROUND OF THE INVENTION

Systems engineers are required to provide a design (e.g. chemical formulation design, engineering design, and the like), which balances conflicting objectives and, at the same time, satisfy multiple constraints and/or requirements. Examples of such objectives in functional materials or drug formulations may include properties (e.g. melting point, solubility in water, viscosity, etc.), manufacturing and development costs, toxicity, compatibility, and the like.

As systems engineers consider many implementation aspects, many design parameters may need to be explored in the process of reaching an optimal design. Non-limiting examples of such design parameters for functional materials or drug formulations may include: chemical recipes (e.g. raw materials and fractional concentrations of the raw materials), process conditions, and optional features. Each possible design may be defined as a configuration (or combination) of design parameter values. The systems engineer needs to find the best design configuration, which optimizes the objectives described above.

Typically, there are also many conflicting objectives to be explored in the design process. For example, objectives in functional materials or drug formulations may include more than thirty properties (density, viscosity, particle size distribution, etc.), and further objectives such as manufacturing and development costs, compatibility and the like. The multiple conflicting objectives may complicate the system optimization procedure.

### SUMMARY OF THE INVENTION

There may be a need to improve a system optimization procedure for generating a recipe profile of a chemical mixture.

The object of the present invention is solved by the subject-matter of the independent claims, wherein further embodiments are incorporated in the dependent claims. It should be noted that the following described aspects of the invention apply also for the computer-implemented method, the device, the computer program element, and the computer readable medium.

According to a first aspect of the present invention, there is provided a computer-implemented method for providing a multi-objective optimal design. The method comprises the steps of:
a) providing (110), via an input channel, a system model for chemical formulation that associates a set of design parameters with a plurality of objective parameters that represent design characteristics of the chemical mixture, wherein the set of design parameters comprises a chemical mixture recipe having two or more ingredients, wherein the plurality of objective parameters comprises two or more physicochemical properties of the chemical mixture;
b) defining (120), via the input channel, a set of primary optimization objective parameters, wherein the set of primary optimization objective parameters comprises one or more essential physicochemical properties of the chemical mixture;
c) performing (130), by a processor, a multi-objective optimizing process on the system model by exploring a plurality of design configurations by assigning specified values to the set of design parameters, such that the set of primary optimization objective parameters meets a specified system requirement and a design goal over a set of defined constrains, by which the range of at least one of the design parameters is limited;
d) determining (140), by the processor, if the multi-objective optimizing process yields a degenerated multi-objective optimal design;
e) if it is determined that the multi-objective optimizing process yields a degenerated multi-objective optimal design, performing (150) a further multi-objective optimizing process on the system model using the set of primary optimization objective parameters and at least one secondary optimization objective parameter to provide a multi-objective optimal design, wherein the at least one secondary optimization objective parameter comprises one or more optional physicochemical properties of the chemical mixture; and
f) providing (160), via an output channel, the multi-objective optimal design that comprises a recipe profile preferably usable for production of the chemical mixture.

In practice, model based prediction is often carried out by models including a dimension reduction algorithm. Examples are principal component regression or partial least squares models. In these models, so-called latent variables are formed and the target variables, which later serve as optimization objectives are modelled as functions of these latent variables rather than as functions of the original input variables, such as chemical composition or process parameters. If the dimension of the space defined by the latent variables is smaller than the dimension of the space defined by the original input parameter for the considered system, then the model exhibits the feature of dimension reduction, and we call the model degenerated. This degeneracy has technical consequences. If a real-world-system is described by a degenerated model with sufficiently high accuracy, then it is possible to systematically change the original input parameters, such as chemical recipe or process, in such a way, that none of the target variables of interest are significantly changed.

The space defined by the set of all possible accessible points in input space with this property is referred to as the “invariant subspace”.

In the proposed approach, statistical optimality principles on the invariant subspace are combined with applied constrains on the original input parameter space to obtain a set of input parameters, e.g. chemical recipes for a chemical formulation design. The input parameters may also be referred to as design parameters and are determined in such a way, that the obtained set of input parameters exhibit optimal statistical variability and simultaneously the target variables of interest (i.e. optimization objective parameters) exhibit theoretically zero or in practice technically only minimal variability. The results generated by the proposed approach can be used to cover not only one or just a few lead-recipes, but the entire class of recipes covered by the invariant subspace. This will be explained hereafter and in particular with respect to the embodiment illustrated in FIG. 1.

The plurality of objective parameters may comprise various chemical and physical properties of the chemical mixture. Take drug formulations as an example, examples of the physicochemical properties of a drug formulation may include, but are not limited to, melting point; permeability across biological or artificial lipid membranes; solubility in water, solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactibility; hygroscopicity; water content (e.g., loss on drying); concentration of impurities; hardness, chemical resistance, color stain resistance, glass transition temperature; and/or other any other chemical, physicochemical and/or physical properties. In some examples, if the chemical mixture is diluted, the physicochemical properties of the chemical mixture may refer to the physicochemical characteristics of the complete mixture (e.g. herbicide formulation plus adjuvant).

The plurality of objective parameters may be divided into a set of primary optimization objective parameters and a set of secondary optimization objective parameters. The set of primary optimization objective parameters comprises one or more essential physicochemical properties of the chemical mixture. The set of primary optimization objective parameters may be provided by a user via a user interface. The set of primary optimization objective parameters may be derived from essential physicochemical properties of similar chemical mixture products. The set of secondary optimization objective parameters comprises one or more optional physicochemical properties of the chemical mixture.

The recipe profile provided in the multi-objective optimal design may represent a chemical mixture with the desired physicochemical properties including the essential physicochemical properties and one or more optional physicochemical properties. In this way, various ingredients may be checked objectively to validate customer requirements of desired physicochemical properties of a chemical mixture, to validate recipes before production/delivery and to tailor chemical products to the needs of customers. Thus, the evaluation does not rely on the subjective impact for test persons or other experimental data.

The proposed computer-implemented method may be suitable for tailoring physicochemical properties of a chemical mixture based on customer's needs. For example, it is possible to modify the secondary optimization objective parameters such that the generated recipe profile has performance characteristics meeting some optional but preferred physicochemical properties.

The proposed computer-implemented method may also be used for exchanging ingredients in a chemical mixture, which may be blocked due to regulatory issues in different countries or lack of resources.

In an example, an iterative approach may be used for performing the optimization. For example, the iteration may be an iterative optimization of single secondary objectives. For example, the iterative approach may comprise a sequence of true Pareto optimizations. This will be explained hereafter and in particular with respect to the embodiment illustrated in FIG. 2.

In another example, a parallel approach may be used for performing Pareto optimization. This will be explained hereafter and in particular with respect to the embodiment illustrated in FIG. 3.

According to an embodiment of the present invention, the set of design parameters further comprises a process condition for producing the chemical mixture.

Many times the properties of mixtures depend on process conditions in addition to the mixture components. For example, environmental variables may influence a property measurement. In the coating example, the temperature of the mixture during measurement can influence the measurement of viscosity. Therefore, process variables, such as temperature, may also be included as a design parameter.

According to an embodiment of the present invention, the computer-implemented method further comprises:
repeatedly performing steps c) to e), until it is determined that the multi-objective optimizing process yields a non-degenerated multi-objective optimal design; and
providing, via an output channel, the non-degenerated multi-objective optimal design.

In this embodiment, an iterative approach is applied to perform Pareto optimization. In other words, Pareto optimization as set of multiple secondary objectives is solved sequentially. This will be explained hereafter and in particular with respect to the embodiment illustrated in FIG. 2.

According to an embodiment of the present invention, step e) further comprises:
performing a further multi-objective optimizing process on the system model using the set of primary optimization objective parameters and a set of multiple secondary objectives defining a Pareto optimization task, for which a Pareto frontier is computed and stored;
providing a user interface allowing a user to interactively navigate along the Pareto frontier based on the set of multiple secondary objectives; and
determining a candidate design from designs calculated in response to the interactive navigation that fulfils the optimality conditions of the set of multiple secondary objectives set by the user via the user interface.

In this embodiment, a parallel approach is applied to perform Pareto optimization. In other words, Pareto optimization as set of multiple secondary objectives is solved simultaneously. This will be explained hereafter and in particular with respect to the embodiment illustrated in FIG. 3.

Optionally, the candidate design may be an interpolation of multiple candidate designs.

According to an embodiment of the present invention, step c) further comprises:
providing a user interface allowing a user to interactively navigate along the Pareto frontier based on the set of primary objectives; and
determining a candidate design from designs calculated in response to the interactive navigation that fulfils the optimality conditions of the set of the primary objectives set by the user via the user interface.

In this embodiment, the generated primary optimal design may result from a selection from a Pareto frontier by a decision maker.

According to an embodiment of the present invention, the chemical mixture comprises one or more of: paint formulation, agricultural multi-component mixture, pharmaceutical multi-component mixture, nutrition multi-component mixture, ink multi-component mixture, chemical mixture for construction purposes, and chemical mixture used inside oil production.

In an example, the multi-objective optimizing process is a Pareto optimization.

The Pareto optimality refers to situations in which it is impossible to make improvement in one parameter without necessarily making it worse in another parameter terms. Given a set of choices and a way of valuing them, the Pareto frontier or Pareto set is the set of choices that are Pareto efficient. In system design, by restricting attention to the set of choices that are Pareto-efficient, a designer can make trade-offs within this set, rather than considering the full range of every parameter.

In some examples, the system model comprises a linear model or a nonlinear model including at least one dimension reduction step.

In an example, also models without explicit algorithmic dimension reduction step may be degenerated, e.g. if it is a priori known that at least one objective is not dependent on at least one input parameter which is then excluded from the model “by hand” during the model building procedure.

In an example, the nonlinear model or the linear model comprises at least one of:
linear regression;
principal component regression;
partial least squares regression;
ridge regression;
a lasso model;
a model whose mathematical form is given by polynomials;
a model whose mathematical form is given by a linear combination of arbitrary ansatz functions;
a model whose mathematical form is given by polynomials of first or second order;
a model whose mathematical form is given by polynomials whose coefficients are determined by at least one of the following algorithms: linear regression, principal component regression, partial least square regression, ridge regression, lasso or any combination thereof;
a model whose mathematical form is given by polynomials of first or second order whose coefficients are determined by at least one of the following algorithms: linear regression, principal component regression, partial least square regression, ridge regression, lasso or any combination thereof;
a parametric model;
a nonparametric model;
a model built on a previous dimension reduction step; or
a model based on any of the above listed techniques applied on an experimental data set.

Examples of the arbitrary ansatz functions may include, but are not limited to, sine and cosine functions as appearing in Fourier Series, exponential functions as appearing as basis for complete monotonic functions, Gaussian Functions, Bessel Functions, Spherical Harmonics, logarithmic functions, rational functions, etc. or any algebraic expression formed from these, whose coefficients are determined by at least one of the following algorithms: linear regression, principal component regression, partial least square regression, ridge regression, lasso, or any combination thereof.

Examples of the parametric model may include, but are not limited to, polynomial regression models or neural network models.

Examples of the model built on a previous dimension reduction step may include, but are not limited to, Feature selection, Feature projection, Feature extraction, Principal component analysis (PCA), Non-negative matrix factorization, Kernel PCA, Graph-based kernel PCA, Linear discriminant analysis, Generalized discriminant analysis, Autoencoder, T-distributed Stochastic Neighbor Embedding, Uniform manifold approximation and projection, K-nearest neighbors algorithm, canonical-correlation analysis, low-dimensional embedding, fast approximate K-NN search, locality sensitive hashin, random projection, Multilinear subspace learning, Multilinear principal component analysis, Multilinear independent component analysis, Multilinear linear discriminant analysis, Multilinear canonical correlation analysis, Independent component analysis, Isomap, Kernel PCA, Latent semantic analysis, Partial least squares, Principal component analysis, Multifactor dimensionality reduction, Nonlinear dimensionality reduction, Multilinear Principal Component Analysis, Multilinear subspace learning, Semidefinite embedding, or Autoencoder.

Examples of the nonparametric model may include, but are not limited to, Spline Interpolation, Gaussian Process Models, Multivariate Adaptive Regression Spline, or Kernel Regression.

Examples of a model based on any of the above-listed techniques applied on an experimental data set may include models based on any of the above-listed techniques applied on an experimental set generated by a Design of Experiments approach, such as, but not limited to, full factorial designs, fractional factorial designs, D-Optimal designs, etc.

One person skilled in the art would appreciate that the nonlinear model or the linear model may comprise a model defined by combinations and/or functions and/or chains of the models, functions, and/or algorithms listed above.

In some examples, the at least one secondary optimization objective parameter comprises at least one of:
an optional objective parameter;
an objective parameter used in means of a D-Optimal design to explore a maximal variability in the set of design parameters leading to the set of improved primary optimization objective parameters; or
an objective parameter including at least one of a determinant, a trace, an eigenvalue, a condition number, or any norm derived from at least one of:
a Fisher information Matrix;
a transpose of the Fisher Information Matrix;
an inverse of the Fisher Information Matrix; or
any combinations thereof to be used in means of an optimal design to explore a maximal variability in the set of design parameters leading to the set of improved primary optimization objective parameters.

For example, the optimal design may be preferentially in form of: A-Optimality, or C-Optimality, or D-Optimality, or E-Optimality, or T-Optimality, or G-Optimality, or I-Optimality, or V-Optimality.

For example, the objective parameter used in means of a D-Optimal design may be the determinant of the Fisher Information Matrix.

In some examples, the system model comprises at least one of:
a system model for modeling chemical processes;
a logistics system model;
an energy system model; and
an engineering system model.

According to a second aspect of the present invention, there is provided a device for providing a multi-objective optimal design. The device comprises an input unit, a processing unit, and an output unit. The input unit is configured to receive a system model that associates a set of design parameters with a plurality of objective parameters that represent design characteristics of a system and a definition of a set of primary optimization objective parameters. The processing unit is configured to (i) perform a multi-objective optimizing process on the system model by exploring a plurality of design configurations by assigning specified values to the set of design parameters, such that the set of primary optimization objective parameters meets a specified system requirement and a design goal over a set of defined constrains; (ii) determine if the multi-objective optimizing process yields a degenerated multi-objective optimal design; and (iii) if it is determined that the multi-objective optimizing process yields a degenerated multi-objective optimal design, perform a further multi-objective optimizing process on the system model using the set of primary optimization objective parameters and at least one secondary optimization objective parameter to provide a multi-objective optimal design. The output unit is configured to provide the multi-objective optimal design.

In some examples, the processing unit is further configured to repeatedly perform steps (i) to (iii) until it is determined that the multi-objective optimizing process yields a non-degenerated multi-objective optimal design. The output unit is configured to provide the non-degenerated multi-objective optimal design.

In this example, the processing unit is configured to use an iterative approach to perform Paetro optimization.

IN some examples, the processing unit is further configured to:
perform a further multi-objective optimizing process on the system model using the set of primary optimization objective parameters and a set of multiple secondary objectives defining a Pareto optimization task, for which a Pareto frontier is computed and stored;
provide a user interface allowing a user to interactively navigate along the Pareto frontier based on the set of multiple secondary objectives; and
determine a candidate design from designs calculated in response to the interactive navigation that fulfils the optimality conditions of the set of multiple secondary objectives set by the user via the user interface.

In this example, the processing unit is configured to use a parallel approach to perform Paetro optimization.

In some examples the processing unit is further configured to:
provide a user interface allowing a user to interactively navigate along the Pareto frontier based on the set of primary objectives; and
determine a candidate design from designs calculated in response to the interactive navigation that fulfils the optimality conditions of the set of the primary objectives set by the user via the user interface.

In this example, the generated primary optimal design results from a selection from a Pareto frontier by the decision maker.

In an example, the multi-objective optimizing process is a Pareto optimization.

In some examples, the system model comprises a linear model or a nonlinear model including at least one dimension reduction step.

In an example, the nonlinear model or the linear model comprises at least one of:
linear regression;
principal component regression;
partial least squares regression;
ridge regression;
a lasso model;
a model whose mathematical form is given by polynomials;
a model whose mathematical form is given by a linear combination of arbitrary ansatz functions;
a model whose mathematical form is given by polynomials of first or second order;
a model whose mathematical form is given by polynomials whose coefficients are determined by at least one of the following algorithms: linear regression, principal component regression, partial least square regression, ridge regression, lasso or any combination thereof;
a model whose mathematical form is given by polynomials of first or second order whose coefficients are determined by at least one of the following algorithms: linear regression, principal component regression, partial least square regression, ridge regression, lasso or any combination thereof;
a parametric model;
a nonparametric model;
a model built on a previous dimension reduction step; or
a model based on any of the above listed techniques applied on an experimental data set.

One person skilled in the art would appreciate that the nonlinear model or the linear model may comprise a model defined by combinations and/or functions and/or chains of the models, functions, and/or algorithms listed above.

In some examples the secondary objective parameters comprises at least one of:
an optional objective parameter;
a objective parameter used in means of a D-Optimal design to explore a maximal variability in the set of design parameters leading to the set of improved primary optimization objective parameters; or
an objective parameter including at least one of a determinant, a trace, an eigenvalue, a condition number, or any norm derived from at least one of:
a Fisher information Matrix;
a transpose of the Fisher Information Matrix;
an inverse of the Fisher Information Matrix; or
any combinations thereof to be used in means of an optimal design to explore a maximal variability in the set of design parameters leading to the set of improved primary optimization objective parameters.

For example, the optimal design may be preferentially in form of: A-Optimality, or C-Optimality, or D-Optimality, or E-Optimality, or T-Optimality, or G-Optimality, or I-Optimality, or V-Optimality.

For example, the objective parameter used in means of a D-Optimal design may be the determinant of the Fisher Information Matrix.

According to a second aspect of the present invention, there is provided a method for monitoring production of a chemical mixture, the method comprising the steps of:
providing a plurality of target objective parameters that represent design characteristics of the chemical mixture;
providing a performance characteristic of a produced chemical mixture that has a recipe profile generated according to the method of any one of the preceding claims; and
comparing the performance characteristic with the design characteristics of the chemical mixture to determine if the produced chemical mixture fulfils predetermined quality criteria.

A comparison between the measured performance characteristics and the design characteristics of the chemical mixture allows not only for quality control or more reliable production but may be extended via a feedback loop which adjusts the production process, where needed.

This will be explained in detail hereinbelow and in particular with respect to the embodiments shown in FIGS. 5 and 7.

According to a third aspect of the present invention, there is provided a method for validating production of a chemical mixture, the method comprising the steps of:
providing an existing performance characteristic for a chemical mixture that has been produced from validated precursors;
generating a recipe profile based on the existing performance characteristic according to the method of any one of claims 1 to 6, wherein the recipe profile comprises an ingredient identifier and related property data, which are associated with at least one new precursor; and
comparing a performance characteristic of a chemical mixture produced using the recipe profile and the existing performance characteristic to validate the at least one new precursor.

This will be explained in detail hereinbelow and in particular with respect to the embodiments shown in FIGS. 6 and 8.

According to a fourth aspect of the present invention, there is provided an apparatus for generating a recipe profile of a chemical mixture, the apparatus comprising one or more processing unit(s) configured to generate the recipe profile of the chemical mixture, wherein the processing unit(s) include instructions, which when executed on the one or more processing unit(s) execute the method steps of the first aspect and any associated example.

According to a fifth aspect of the present invention, there is provided an apparatus for monitoring production of a chemical mixture, the apparatus comprising one or more processing unit(s) configured to monitor production, wherein the processing unit(s) include instructions, which when executed on the one or more processing unit(s) execute the method steps of the second aspect and any associated example.

According to a sixth aspect of the present invention, there is provided an apparatus for validating production of a chemical mixture, the apparatus comprising one or more processing unit(s) configured to validate production, wherein the processing unit(s) include instructions, which when executed on the one or more processing unit(s) execute the method steps of the third aspect and any associated example

According to another aspect of the present invention, there is provided a computer program element comprising instructions, which when executed by a processing unit, cause the processing unit to carry out the steps of the method of the first, second, or third aspect and any associated example.

According to a further aspect of the present invention, there is provided a computer readable medium having stored the program element.

As used herein, the term “unit” may refer to, be part of, or include an Application Specific Integrated Circuit (ASIC), an electronic circuit, a processor (shared, dedicated, or group) and/or memory (shared, dedicated, or group) that execute one or more software or firmware programs, a combinational logical circuit, and/or other suitable components that provide the described functionality.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart that illustrates a computer-implemented method according to some embodiments of the present disclosure.

FIG. 2 is a flowchart that illustrates a computer-implemented method according to some further embodiments of the present disclosure.

FIG. 3 is a flowchart that illustrates a computer-implemented method according to some further embodiments of the present disclosure.

FIG. 4 schematically illustrates a device according to some embodiments of the present disclosure.

FIG. 5 shows an example of a flowchart for monitoring quality of the chemical mixture in a production process of the chemical mixture having a target design characteristics.

FIG. 6 shows an example of a flowchart for validating the production of the chemical mixture.

FIG. 7 shows an example of a production line for producing the chemical mixture with a monitoring apparatus.

FIG. 8 shows another example of a production line for producing the chemical mixture with a validation apparatus.

### DETAILED DESCRIPTION OF EMBODIMENTS

According to a first aspect of the present disclosure, there is provided a computer-implemented method 100 for generating a recipe profile of a chemical mixture. The method 100 comprises the steps of:
a) providing 110, via an input channel, a system model for modelling the chemical formulation that associates a set of design parameters with a plurality of objective parameters that represent design characteristics of a system, wherein the set of design parameters comprises a chemical mixture recipe having two or more ingredients, wherein the plurality of objective parameters comprises two or more physicochemical properties of the chemical mixture;
b) defining 120, via the input channel, a set of primary optimization objective parameters, wherein the set of primary optimization objective parameters comprises one or more essential physicochemical properties of the chemical mixture;
c) performing 130, by a processor, a multi-objective optimizing process on the system model by exploring a plurality of design configurations by assigning specified values to the set of design parameters, such that the set of primary optimization objective parameters meets a specified system requirement and a design goal over a set of defined constrains, by which the range of at least one of the design parameters is limited;
d) determining 140, by the processor, if the multi-objective optimizing process yields a degenerated multi-objective optimal design;
e) if it is determined that the multi-objective optimizing process yields a degenerated multi-objective optimal design, performing 150 a further multi-objective optimizing process on the system model using the set of primary optimization objective parameters and at least one secondary optimization objective parameter to provide a multi-objective optimal design, wherein the at least one secondary optimization objective parameter comprises one or more optional physicochemical properties of the chemical mixture; and
f) providing 160, via an output channel, the multi-objective optimal design that comprises a recipe profile preferably usable for production of the chemical mixture.

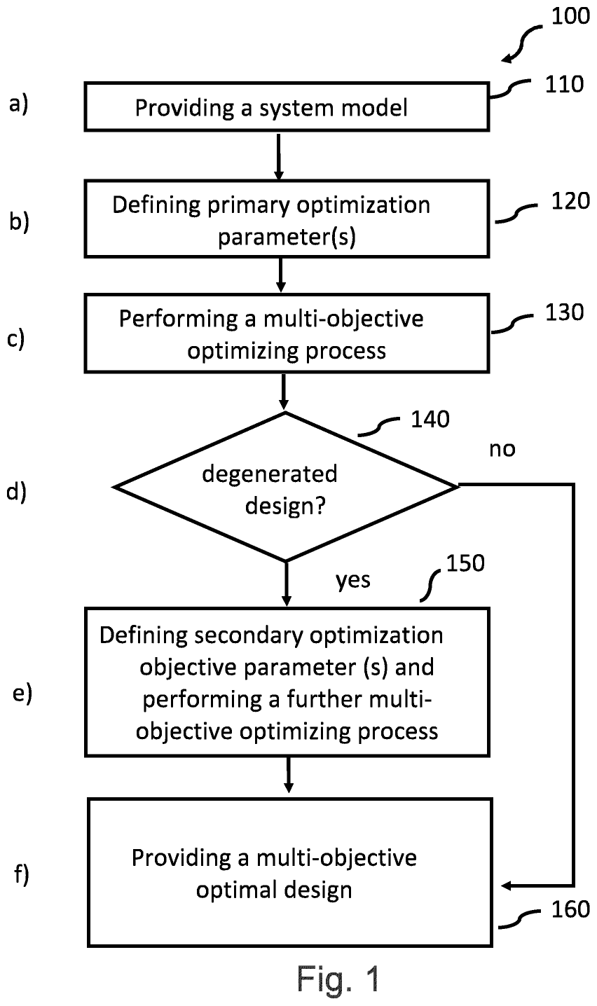

FIG. 1 is a flowchart that illustrates an example of the computer-implemented method 100 according to the first aspect of the present disclosure.

The computer-implemented method 100 may be implemented as a device, module or related component in a set of logic instructions stored in a non-transitory machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof. For example, computer program code to carry out operations shown in the method 100 may be written in any combination of one or more programming languages, including an object oriented programming language such as JAVA, SMALLTALK, C++, Python, or the like and conventional procedural programming languages, such as the “C” programming language or similar programming languages.

### System Model

In step 110, i.e. step a), a system model is provided via an input channel. The system model associates a set of design parameters with a plurality of objective parameters that represent design characteristics of a system. The system model is a system model for modelling chemical formulations. The set of design parameters comprises a chemical mixture recipe having two or more ingredients. The plurality of objective parameters comprises two or more physicochemical properties of the chemical mixture.

Depending on the type of chemical formulations, the system model may comprise a linear model. Alternatively, the system model may comprise a nonlinear model including at least one dimension reduction step.

Examples of the linear or nonlinear model may include, but are not limited to,
linear regression;
principal component regression;
partial least squares regression;
ridge regression;
a lasso model;
a model whose mathematical form is given by polynomials;
a model whose mathematical form is given by a linear combination of arbitrary ansatz functions, such as, but not limited to, sine and cosine functions as appearing in Forier Series, exponential functions as appearing as basis for complete monotonic functions, Gaussian Functions, Bessel Functions, Spherical Harmonics, logarithmic functions, rational functions, etc. or any algebraic expression formed from these functions;
a model whose mathematical form is given by polynomials of first or second order;
a model whose mathematical form is given by polynomials whose coefficients are determined by at least one of the following algorithms: linear regression, principal component regression, partial least square regression, ridge regression, lasso or any combination thereof;
a model whose mathematical form is given by polynomials of first or second order whose coefficients are determined by at least one of the following algorithms: linear regression, principal component regression, partial least square regression, ridge regression, lasso or any combination thereof;
a model whose mathematical form is given by a linear combination of arbitrary ansatz functions, such as, but not limited to, sine and cosine functions as appearing in Forier Series, exponential functions as appearing as basis for complete monotonic functions, Gaussian Functions, Bessel Functions, Spherical Harmonics, logarithmic functions, rational functions, etc. or any algebraic expression formed from these functions, whose coefficients are determined by at least one of the following algorithms: linear regression, principal component regression, partial least square regression, ridge regression, lasso or any combination thereof;
any kind of parametric model, such as, but not limited to, polynomial regression models and neural network models;
a nonparametric models, such as, but not limited to, Spline Interpolation, Gaussian Process Models, Multivariate Adaptive Regression Spline, Kernel Regression;
any model, including any one of the explicit possibilities above, built on a previous dimension reduction step, such as, but not limited to, Feature selection, Feature projection, Feature extraction, Principal component analysis (PCA), Non-negative matrix factorization, Kernel PCA, Graph-based kernel PCA, Linear discriminant analysis, Generalized discriminant analysis, Autoencoder, T-distributed Stochastic Neighbor Embedding, Uniform manifold approximation and projection, K-nearest neighbors algorithm, canonical-correlation analysis, low-dimensional embedding, fast approximate K-NN search, locality sensitive hashin, random projection, Multilinear subspace learning, Multilinear principal component analysis, Multilinear independent component analysis, Multilinear linear discriminant analysis, Multilinear canonical correlation analysis, Independent component analysis, Isomap, Kernel PCA, Latent semantic analysis, Partial least squares, Principal component analysis, Multifactor dimensionality reduction, Nonlinear dimensionality reduction, Multilinear Principal Component Analysis, Multilinear subspace learning, Semidefinite embedding, Autoencoder;
any model based on any of the above listed techniques applied on an experimental data set;
any model based on any of the above listed techniques applied on an experimental data set generated by a Design of Experiments approach, such as, but not limited to full factorial designs, fractional factorial designs, D-Optimal designs; or
a model defined by combinations and/or functions and/or chains of the models, functions, and/or algorithms listed above.

In the following, x₁, x₂, . . . , xₙ represent the set of design parameters of the considered system model. The set of design parameters may be e.g. original control and/or input parameters of the considered system.

Further, y₁, y₂, . . . , yₖ represent the plurality of objective parameters of the considered system model. Examples of the objective parameters may be e.g. response and target variables of interest.

The range of the design parameters, i.e. input variables, is typically limited by a set of constrains. Both linear and nonlinear functions may be covered. Typically, but not limited to this case, we consider linear constrains of at least one of the following forms:

al₁·x₁+al₂·x₂+ . . . +alₙ·xₙ>cl; (a)

al₁·x₁+al₂·x₂+ . . . +alₙ·xₙ<cl; or (b)

al₁·x₁+al₂·x₂+ . . . +alₙ·xₙ=cl. (c)

The coefficients al and cl are arbitrary numbers. Typically, but not restricted to this case, the defined constrains may encode prior knowledge of a system expert, and may be based, but not limited to, both on practical experience and theoretical considerations such as fundamental, empirical, or semi-empirical physical, chemical, or technical formulas.

Further, z₁, z₂, . . . , zₘ with m<n represent a set of “latent variables”, such as, but not limited to, results from a principal components analysis (PCA) based on x₁, . . . , xₙ, or latent variables of a partial least squares (PLS) regression model connecting x₁, . . . , xₙ, and y₁, . . . , yₖ via the latent variables z₁, . . . , zₘ.

We assume a validated model, such as, but not limited to, a PCA or PLS regression, that can with sufficiently accuracy predict y₁, . . . , yₖ from x₁, . . . , xₙ via z₁, . . . , zₘ;

x₁, . . . ,xₙ=>z₁, . . . ,zₘ=>y₁, . . . ,yₖ.

Generalizations to arbitrary nonlinear models including a dimension reduction steps shall also be covered, such as, but not limited to auto-encoders, neural networks, and the like.

We assume that m<n, i.e. that the model is degenerated.

The system model may comprise at least one of the following models.

A. Chemical Formulations

In an example, the system model may be used to model chemical formulations, e.g. for predicting the properties of a chemical mixture. Examples of the chemical mixture may include, but are not limited to, paint formulation, agricultural multi-component mixture, pharmaceutical multi-component mixture, nutrition multi-component mixture, ink multi-component mixture, chemical mixture for construction purposes, and chemical mixture used inside oil production.

The design parameters, i.e. input variables, of the model for predicting the properties of a chemical mixture may include chemical mixture recipes having two or more ingredients. In some examples, a single chemical mixture recipe may comprise up to fifty different raw materials, i.e. ingredients. The two or more ingredients are expressed as fractional concentrations of the total amount of the chemical mixture. In general, the property of a chemical mixture depends on the ingredient component fractional concentrations rather than the total amount of the chemical mixture. Mixture formulas may be expressed in weight, volume, or other quantity units, such as the relative concentration of reactive groups per monomer type if mixtures of monomers with different amount of functional groups per monomer are considered. The fractional concentration is simply the quantity of an ingredient in the chemical mixture divided by the total quantity of the mixture. The sum of the fractional concentrations will be unity. Fractional concentrations are continuous variable in the range between 0 and 1.

Many times the properties of mixtures depend on process conditions in addition to the mixture components. For example, environmental variables may influence a property measurement. In the coating example, the temperature of the mixture during measurement can influence the measurement of viscosity. Therefore, process variables, such as temperature, may also be included as a design parameter.

The plurality of objective parameters of the model for predicting the properties of a chemical mixture may comprise properties of the chemical mixture. Properties of the chemical mixture may be any measurable characteristic. The characteristic may be a continuous, ordinal, or nominal measurement. For example, a formulated coating could have a measurement of the viscosity of the liquid mixture on a continuous scale. For example, the measurement of orange peel of the applied coating film may be on a decimal category ordinal scale from 1 (very unsmooth) to 10 (very smooth). In another example, the properties of each chemical mixture recipe further comprise, for each measured property, a respective performance score indicative of a performance evaluation of the respective chemical mixture recipe, e.g. from 1 (very good) to 5 (very bad). An example of a nominal measurement may be the coded categories of pass or fail for observation of some defect.

In the following, exemplary design parameters (e.g. raw materials) and exemplary objective parameters (e.g. properties) for various chemical mixtures will described.

1. Agricultural Multi-Component Mixtures

For example, there are mixtures used for agricultural purposes like formulations used as sprays for treating crops with insecticides, fungicides and so on. Thereby on the one side the sprayability of the active ingredients is guaranteed by the residual components inside the formulation. I.e. the different other components of the formulation besides the active ingredient are used to obtain a formulation, which is applicable under the given process of spraying. I.e. the sprayability (e.g. droplet size formation, ease of forming such a droplet and so on) might be properties, which are influenced by the different components of such a formulation together with the nature of the active ingredient.

Furthermore, also the adsorption of the sprayed formulation on the plant and the absorption, which is resorption in this context, of the active ingredient or complete sprayed formulation are depending on the active ingredient and the residual components in the formulation. Moreover, also the target-oriented way—or better said movement of the active ingredient to the targeted part of the cell—of the active ingredient inside a plant/organism will be influenced by the residual components inside such a formulation. I.e. the speed of effect generation and the effect generation itself are depending on these shares of the formulation.

2. Pharmaceutical Multi-Component Mixtures

Also, here the components being present in a pharmaceutical formulation besides the active ingredient influence the complete lifecycle of such a pharmaceutical—herein, from preparation to excretion or “digestion”.

For example, these formulation shares define, whether an active ingredient is provided as pill, suppositories or as a liquid, which mostly is a dispersion of the active ingredient.

Furthermore, these formulation shares define, where inside an organism the active ingredient is set free and where it can be absorbed respectively resorbed.

Finally, these formulation shares define, to which parts inside a body respectively cell the active ingredient is transported and there digested to show the wished effect; or, if it is not “digested” inside the organism at all and excreted without “digestion”.

Each of these properties may be important to find the right formulation, i.e. composition of the pharmaceutical multi-component mixtures.

3. Nutrition Multi-Component Mixtures

Many foods can be looked at as multi-component mixtures comprising different kind of chemical sub-groups necessary for our organisms to work properly. Nutrition additives like e.g. vitamins, mineral nutrients and so on are a part of foods also, whereby it is important to integrate these into these food “formulations” in a way that these are available at the right parts of the organism. Again, both parameters can be influenced by the residual shares of the food “formulations”. For example, the right way of offering mineral nutrients to an organism can guarantee a good resorption by the organism, whereas a worse way of offering can reduce the resorption, what then can cause health effects.

4. Inks as Multi-Component Mixtures

Similar, inks are also multi-component mixtures, i.e. they can be defined as ink formulations also. Also, here the residual components beside the colour providing ingredients—in this case mostly dyes—guarantee the stability of the ink, the process-ability and the fixation on the to-be-inked surface.

Here, the properties being of specific importance, are properties like adhesion to the to-be-inked surface, sagging resistance or viscosity stability of the formulation after application and lightfastness of the resulting print, i.e. non-fading of the resulting print.

5. Chemical Mixtures for Construction Purposes

Also, a lot of materials used inside construction applications can be looked at as chemical mixtures. E.g. concrete is formed out of a mixture of cement, rockets of different sizes and water. Furthermore, a modern concrete formulation also contains concrete additions and concrete admixtures, both, additives for these formulations to trigger and tailor-make specific properties of the concrete formulations. Such properties are for example the application behaviour, the settling behaviour, the hardening, the tensile strength, the bending property and the durability of the concrete in wet or in dried form. All these properties can be influenced by concrete additions and concrete admixtures. Whereas the substances used as concrete addition materials are mostly inorganics like e.g. rock flour, fly ash or silica fume, the substances used as concrete admixture materials can also be of organic character, like e.g. acrylics or other oligo- or polymeric substances.

A related application may also be chemical mixtures used as materials for plastering. Thereby, also formulations are used, which are similar to concrete formulations. However, these plaster mortars are usually limited with respect to the size of the rockets. I.e. the rock's aggregate is limited to a size of 4 mm, no bigger sizes are allowed to be used for these mortars. The main properties, which need to be achieved also by the use of the right additives, which are very similar to the ones mentioned above, are mainly in the area of application properties respectively workability. Pumpability, smoothing property, but also adhesion properties are evaluated usually during the development of such plastering formulations.

6. Chemical Mixtures Used Inside Oil Production

Also, in oil production chemical mixtures are used to optimize the degree of efficiency of oil extraction. In fracking and in conventional oil extraction methods, especially at late stages of the lifecycle of a wellbore, the efficiency level is elevated by pumping of these formulations into the wellbore. Thereby, mainly water comprising organic polymers are used. Overall, the efficiency level of oil production is a parameter for the effectiveness of the additives used. In a detail view, properties like the ability to release oil from stones or the ability to generate pressure and viscosity under such conditions might be important properties.

B. Chemical Processes

In an example, the model may be used for modelling chemical processes.

Using the industrial aging processes as an example, aging of critical assets is an omnipresent phenomenon in any production environment, causing significant maintenance expenditures or leading to production losses. The understanding and anticipation of the underlying degradation processes is therefore of great importance for a reliable and economic plant operation, both in discrete manufacturing and in the process industry.

With a focus on the chemical industry, notorious aging phenomena include the deactivation of heterogeneous catalysts due to coking, sintering, or poisoning; plugging of process equipment, such as heat exchangers or pipes, on process side due to coke layer formation or polymerization; fouling of heat exchangers on water side due to microbial or crystalline deposits; erosion of installed equipment, such as injection nozzles or pipes, in fluidized bed reactors; and more.

This understanding has commonly been condensed into sophisticated mathematical models. Examples of such mechanistic degradation models deal with coking of steamcracker furnaces, sintering or coking of heterogeneous catalysts, or crystallization fouling of heat exchangers.

In this example, the design parameters may thus include various process variables, such as temperature, flow rate, pressure, etc., while the objective parameters may include one or more key performance indicators for quantifying the progress of its degradation.

One skilled in the art would understand that the method of the present disclosure is also applicable for other system models, such as logistics system model, energy system models, engineering system models, etc.

### Primary Optimization Objective Parameters

In step 120, i.e. step b), a set of primary optimization objective parameters is defined. The primary optimization objective parameters may be also referred to as primary optimization targets.

The primary optimization objective parameters may be essential objectives to be optimized, while the secondary optimization objective parameters may be preferred or optional objectives to be optimized.

The set of primary optimization objective parameters for chemical mixture may comprise one or more essential physicochemical properties of the chemical mixture, while the secondary optimization objective parameters may be preferred or optional physicochemical properties to be optimized.

For example, in chemical process design two levels of objective criteria are often considered: design criteria and final decision criteria. The first group may contain, e.g. product purities, column duties, and reboil ratios. The second group may comprise hard economic objectives like investment and operating costs, often more softer environmental issues as sustainability key figures and objectives regarding health and safety. Therefore, one or more optimization objective parameters in the first group may represent the primary optimization objective parameters, while one or more optimization objective parameters in the second group may represent the secondary optimization objective parameters.

For example, in the above-described exemplary system model, y₁, . . . , yₖ will be considered as primary optimization objectives.

### Multi-Objective Optimizing Process

As in any engineering problems or projects, these primary objective parameters and constraints are generally in conflict and interact with one another and the design parameters in nonlinear manners. Thus, it may be not very clear how to modify them to achieve the “best” design or trade off.

Thus, in step 130, i.e. step c), a multi-objective optimizing process is performed by a processor on the system model by exploring a plurality of design configurations by assigning specified values to the set of design parameters, such that the set of primary optimization objective parameters meets a specified system requirement and a design goal over a set of defined constrains, by which the range of at least one of the design parameters is limited.

Typically, the multi-objective optimizing process may result in a set of optimal solutions that represent different trade-offs among objectives, i.e. objective parameters. These solutions are also referred to as Pareto optimal solutions or Pareto optimal solution set. Design objective function space representation of the Pareto optimal solution set is known as Pareto optimal front (POF). One strategy to find Pareto optimal solutions is to convert the multi-objective optimization problem to a single objective optimization problem and then find a single trade-off solution.

In an example, the multi-objective optimizing process is based on genetic algorithm, which has been demonstrated to efficiently solve multi-objective optimization problems because they result in diverse set of trade-off solutions in a single numerical simulation.

In an example, the multi-objective optimizing process is based on evolutionary algorithm, such as crossovers and/or mutations, which is used for creating future generations.

For example, in the above-described exemplary system model, a multicriterial optimization algorithm is applied to optimize the values of the above-mentioned primary optimization objectives y₁, . . . , yₖ. The found optimal values are denoted by y₁*, . . . , yₖ*. The individual objectives here may be minimizing, maximizing, or approaching a desired target value while satisfying one of the constrains under section “system model”.

In an example, the multi-objective optimizing process is a Pareto optimization based on the sandwiching or the hyperboxing method as described in Bortz M, Burger J, Asprion N, Blagov S, Böttcher R, Nowak U, Scheithauer A, Welke R, Kufer K-H, Hasse H. Multi-criteria optimization in chemical process design and decision support by navigation on Pareto sets. Computers and Chemical Engineering 2014; 60: 354-363.

In another example, the secondary objective is chosen after selection of a preferred Pareto optimal configuration on from a calculated pareto frontier, preferentially calculated by the sandwiching or the hyperboxing method as described in Bortz M, Burger J, Asprion N, Blagov S, Böttcher R, Nowak U, Scheithauer A, Welke R, Kufer K-H, Hasse H. Multi-criteria optimization in chemical process design and decision support by navigation on Pareto sets. Computers and Chemical Engineering 2014; 60: 354-363.

In a further example, the selection of the Pareto optimal configuration from the pareto frontier is carried out by graphical navigation as described in Bortz M, Burger J, Asprion N, Blagov S, Böttcher R, Nowak U, Scheithauer A, Welke R, Küfer K-H, Hasse H. Multi-criteria optimization in chemical process design and decision support by navigation on Pareto sets. Computers and Chemical Engineering 2014; 60: 354-363.

Optionally, the generated primary optimal design may result from a selection from a Pareto frontier by the decision maker. Accordingly, step c) may further comprise the step of providing a user interface allowing a user to interactively navigate along the Pareto frontier based on the set of primary objectives and the step of determining a candidate design from designs calculated in response to the interactive navigation that fulfils the optimality conditions of the set of the primary objectives set by the user via the user interface. In other words, to support the decision maker, the data may be visualized in a user interface, which allows the decision maker to explore the Pareto set and its trade-offs between the different primary objectives by using graphical controls. Based on this, the design point is selected and optionally re-optimized. For more information concerning visualizing the Pareto set, reference is made to Bortz M, Burger J, Asprion N, Blagov S, Böttcher R, Nowak U, Scheithauer A, Welke R, Küfer K-H, Hasse H. Multi-criteria optimization in chemical process design and decision support by navigation on Pareto sets. Computers and Chemical Engineering 2014; 60: 354-363 and Kufer et al. Multicriteria optimization in intensity modulated radiotherapy planning. In P. M. Pardalos, & H. E. Romeijn (Eds), Handbook of optimization in medicine (pp. 1123-168), Springer.

### Secondary Optimization Objective Parameters

In step 140, i.e. step d), the processor determines whether the multi-objective optimizing process yields a degenerated multi-objective optimal design.

If it is determined that the multi-objective optimizing process does not yield a degenerated multi-objective optimal design, step 160, i.e. step f), is performed. In other words, the multi-objective optimal design is provided via the output channel.

On the other hand, if it is determined that the multi-objective optimizing process yields a degenerated multi-objective optimal design, step 150, i.e. step e), is performed.

For example, in the above-described exemplary system model, we assume that m<n, i.e. that the model is degenerated. Due to the given degeneracy, only the values z₁*, . . . , zₘ* of the latent variables z₁, . . . , zₘ may be uniquely determined corresponding to the optimized values y₁*, . . . , yₖ*, while the back mapping to x₁, . . . , xₙ is not unique.

Thus, in step 150, i.e. step e), a further multi-objective optimizing process is performed on the system model using the set of primary optimization objective parameters and at least one secondary optimization objective parameter to provide a multi-objective optimal design.

For example, we consider a new set of secondary optimization objective parameters yₖ₊₁, . . . , yₖ₊ₗ. We assume that these can be modelled by using the new latent variable representation:

x₁, . . . ,xₙ=>z₁, . . . ,zₘ,zₘ₊₁, . . . ,zₘ₊ⱼ=>yₖ₊₁, . . . ,yₖ₊ₗ

While zₘ₊₁, . . . , zₘ₊ⱼ are newly introduced, zₘ may remain identical.

We fix z₁, . . . , zₘ=z₁*, . . . , zₘ*. A multicriterial optimization algorithm is applied to optimize the values of yₖ₊₁, . . . , yₖ₊ₗ by only varying zₘ₊₁, . . . , zₘ₊ⱼ.

The secondary optimization targets, i.e. the secondary optimization objective parameters, may be the typical ones similar to the primary targets or also may be defined by an optimized experimental design exploring the orthogonal complement of z₁, . . . , zₘ, i.e. in means of a D-Optimal design to explore the maximal variability in x₁, . . . , xₙ leading to the same fixed optimized y₁*, . . . , yₖ*.

Further examples of the secondary optimization objective parameters may include, but are not limited to, an optional objective parameter, or an objective parameter used in means of a D-Optimal design to explore a maximal variability in the set of design parameters leading to the set of improved primary optimization objective parameters, or an objective parameter including at least one of a determinant, a trace, an eigenvalue, a condition number, or any norm derived from at least one of: a Fisher information Matrix, a transpose of the Fisher Information Matrix, an inverse of the Fisher Information Matrix, or any combinations thereof to be used in means of an optimal design to explore a maximal variability in the set of design parameters leading to the set of improved primary optimization objective parameters. For example, the optimal design may be preferentially in form of: A-Optimality, or C-Optimality, or D-Optimality, or E-Optimality, or T-Optimality, or G-Optimality, or I-Optimality, or V-Optimality. For example, the objective parameter used in means of a D-Optimal design may be the determinant of the Fisher Information Matrix. Using drug formulations as an example, the set of primary optimization objective parameters may comprise one or more of melting point; permeability across biological or artificial lipid membranes; solubility in water, solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactibility; hygroscopicity; water content (e.g., loss on drying); concentration of impurities; hardness, chemical resistance, color stain resistance, glass transition temperature; other chemical, physicochemical and/or physical properties; and information on compatibility and stability. The set of secondary optimization objective parameters may comprise one or more of cost, toxicity, and compatibility.

In step 160, i.e. step f), the multi-objective optimal design is provided via an output channel.

With the proposed method, optimization in a design of a chemical mixture) may be focusing on objectives of two levels: the primary optimization objective parameters (i.e. essential physicochemical properties) and the secondary optimization parameters (i.e. optional physicochemical properties). In the design, the systems engineers may firstly try to find an optimal solution in the multi-dimensional objective space with the primary optimization objective parameters by empirical iterative change of the design parameters in the design space. If it is determined that the multi-objective optimizing process yields a degenerated multi-objective optimal design (i.e. the dimension of the space defined by the latent variables is smaller than the dimension of the space defined by the original input parameter for the considered system), one or more secondary optimization objective parameters may be included. The systems engineers then try to find an optimal solution in the multi-dimensional objective space with the primary optimization objective parameters and the one or more secondary optimization parameters by empirical iterative change of the design parameters in the design space.

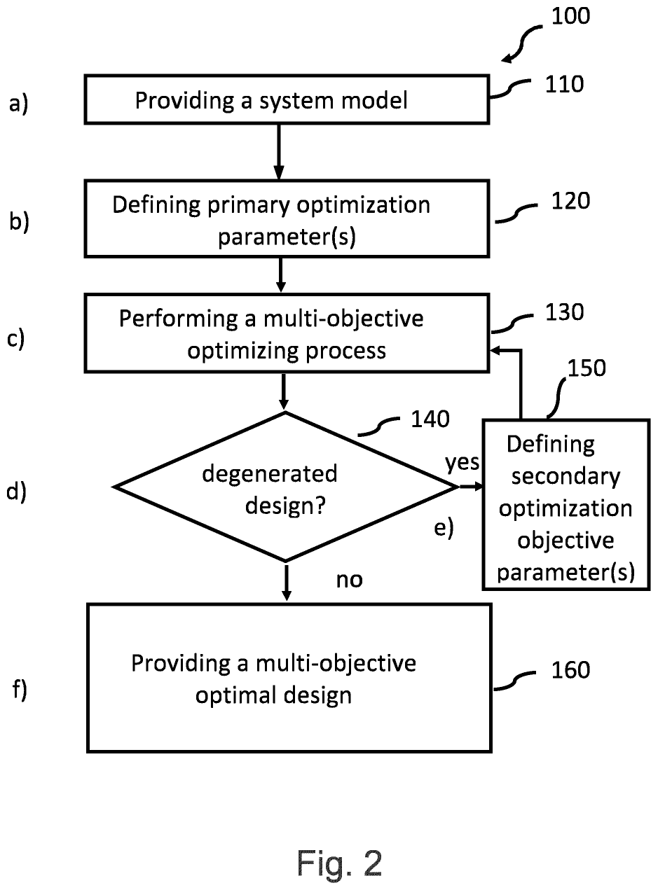

Optionally, as illustrated in FIG. 2, the computer-implemented method may use an iterative approach to perform Pareto optimization. The computer-implemented method in FIG. 2 further comprises the steps of repeatedly performing steps c) to e), until it is determined that the multi-objective optimizing process yields a non-degenerated multi-objective optimal design, and providing, via the output channel, the non-degenerated multi-objective optimal design. In other words, the entire procedure may be iterated, if also the secondary optimization yields a degenerated result.

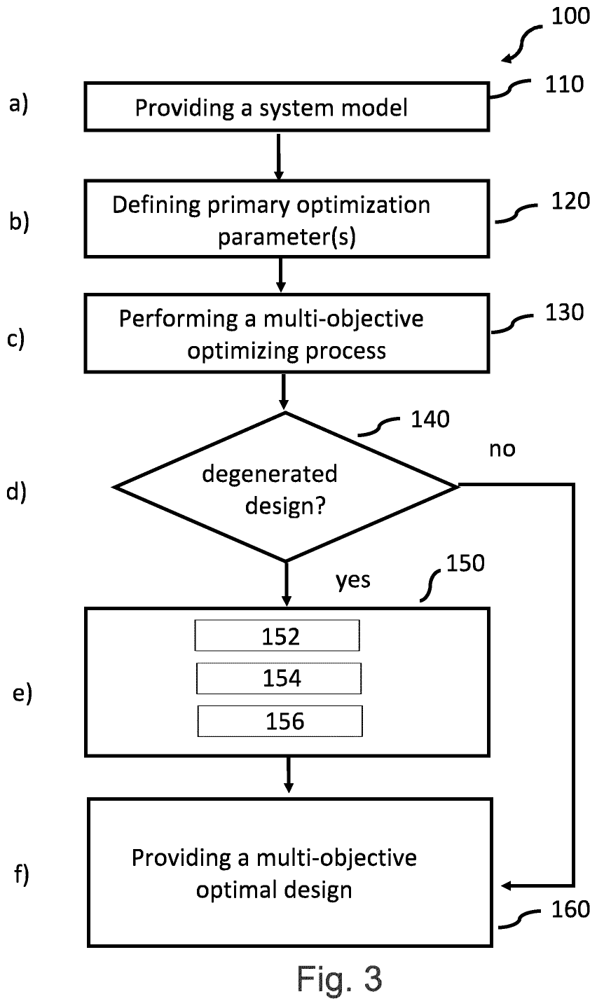

Optionally, as illustrated in FIG. 3, the computer-implemented method may use a parallel approach to perform Pareto optimization.

In step 152, a further multi-objective optimizing process on the system model is performed using the set of primary optimization objective parameters and a set of multiple secondary objectives defining a Pareto optimization task, for which a Pareto frontier is computed and stored. In an example, we assume that significant non-convexities do not occur frequently, and that most regions of the Pareto sets are convex. The basic idea is to use a sandwich approximation method which is able to approximate the convex part of the Pareto set efficiently. Once a certain approximation quality is achieved there, candidate regions for non-convex behavior are identified and tested for non-convexity. Finally, the non-convex regions are sampled using a hyperboxing scheme. The sandwich approximation method creates successively inner and outer approximations to the Pareto set by using a weighted sum scalarization to calculate Pareto points. The weight vectors are the normals to the supporting, tangential hyperplanes of the Pareto set at the calculated points. These hyperplanes represent the outer approximation.

The inner approximation is found from the close-by facets of the convex hull of the Pareto points. New Pareto points are added as long as the difference between outer and inner approximation—the sandwich—is still beyond some arbitrary but fixed threshold, i.e. the desired approximation quality. The basic idea of approximation of the Pareto set is outlined above, details are published in Bortz M, Burger J, Asprion N, Blagov S, Böttcher R, Nowak U, Scheithauer A, Welke R, Kufer K-H, Hasse H. Multi-criteria optimization in chemical process design and decision support by navigation on Pareto sets. Computers and Chemical Engineering 2014; 60: 354-363.

In step 154, a user interface is provided allowing a user to interactively navigate along the Pareto frontier based on the set of multiple secondary objectives. The result of the automated calculations of the hybrid algorithm is a finite set of points, which approximate the Pareto set within a certain accuracy. To support the decision maker, the data are visualized in a user interface which allows the decision maker to explore the Pareto set and its trade-offs between the different secondary objectives by using graphical controls. Based on this, the design point is selected and optionally re-optimized. For more information concerning visualizing the Pareto set, reference is made to Bortz M, Burger J, Asprion N, Blagov S, Böttcher R, Nowak U, Scheithauer A, Welke R, Kufer K-H, Hasse H. Multi-criteria optimization in chemical process design and decision support by navigation on Pareto sets. Computers and Chemical Engineering 2014; 60: 354-363 and Küfer et al. Multicriteria optimization in intensity modulated radiotherapy planning. In P. M. Pardalos, & H. E. Romeijn (Eds), Handbook of optimization in medicine (pp. 1123-168), Springer.

In step 156, a candidate design is determined from designs calculated in response to the interactive navigation that fulfils the optimality conditions of the set of multiple secondary objectives set by the user via the user interface. Optionally, the candidate design may be an interpolation of multiple candidate designs.

It should also be understood that, unless clearly indicated to the contrary, in any methods claimed herein that include more than one step or act, the order of the steps or acts of the method is not necessarily limited to the order in which the steps or acts of the method are recited.

### Multi-Objective Optimal Design Supporting Device

According to a second aspect of the present disclosure, there is provided a device 10 for providing a multi-objective optimal design. The device comprises an input unit 12, a processing unit 14, and an output unit 16.

The input unit 12 is configured to receive a system model that associates a set of design parameters with a plurality of objective parameters that represent design characteristics of a system and a definition of a set of primary optimization objective parameters.

The processing unit 14 is configured to (i) perform a multi-objective optimizing process on the system model by exploring a plurality of design configurations by assigning specified values to the set of design parameters, such that the set of primary optimization objective parameters meets a specified system requirement and a design goal over a set of defined constrains, by which the range of at least one of the design parameters is limited; (ii) determine if the multi-objective optimizing process yields a degenerated multi-objective optimal design; and (iii) if it is determined that the multi-objective optimizing process yields a degenerated multi-objective optimal design, perform a further multi-objective optimizing process on the system model using the set of primary optimization objective parameters and at least one secondary optimization objective parameter to provide a multi-objective optimal design.

The output unit 16 is configured to provide the multi-objective optimal design.

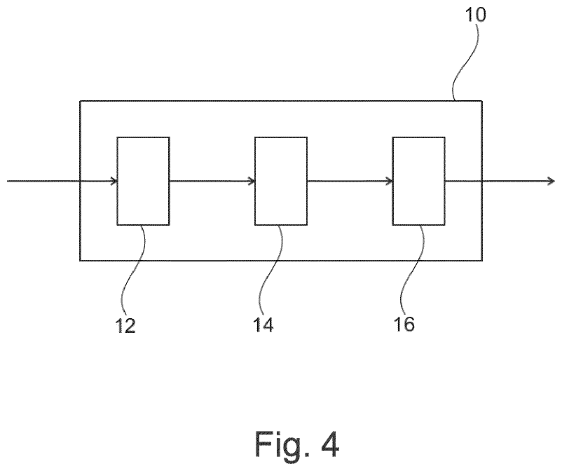

FIG. 4 schematically illustrates an example of the device 10 according to the second aspect of the present disclosure. The device 10 may be implemented as an embedded computing device or on a personal computer, for example.

The input unit 12 is configured to receive a system model that associates a set of design parameters with a plurality of objective parameters that represent design characteristics of a system and a definition of a set of primary optimization objective parameters.

In an example, the system model is a model for modelling chemical formulations and processes. Examples of chemical formulations and processes may include chemical formulations, e.g. in coatings and paints, adhesives, in the field of crop protection and fertilization, in seed treatment, in laundry processes (e.g. in a washing machine, a dishwasher, or an industrial laundry machine), in food (e.g. milk, or meat) processing, in animal feed processing, in biofuel production, in leather production, in textile production, in pulp and paper industry, in beverage production, in chemical production processes, in water treatment, and/or in the field of human and veterinary medicine.

A skilled person will appreciate that the device is also applicable for a logistics system model, an energy system model, an engineering system model, and the like.

The system model may comprise a linear model or a nonlinear model including at least one dimension reduction step. Examples of the linear or nonlinear model may include, but are not limited to:
linear regression;
principal component regression;
partial least squares regression;
ridge regression;
a lasso model;
a model whose mathematical form is given by polynomials;
a model whose mathematical form is given by a linear combination of arbitrary ansatz functions, such as, but not limited to, sine and cosine functions as appearing in Forier Series, exponential functions as appearing as basis for complete monotonic functions, Gaussian Functions, Bessel Functions, Spherical Harmonics, logarithmic functions, rational functions, etc. or any algebraic expression formed from these functions;
a model whose mathematical form is given by polynomials of first or second order;
a model whose mathematical form is given by polynomials whose coefficients are determined by at least one of the following algorithms: linear regression, principal component regression, partial least square regression, ridge regression, lasso or any combination thereof;
a model whose mathematical form is given by polynomials of first or second order whose coefficients are determined by at least one of the following algorithms: linear regression, principal component regression, partial least square regression, ridge regression, lasso or any combination thereof;
a model whose mathematical form is given by a linear combination of arbitrary ansatz functions, such as, but not limited to, sine and cosine functions as appearing in Forier Series, exponential functions as appearing as basis for complete monotonic functions, Gaussian Functions, Bessel Functions, Spherical Harmonics, logarithmic functions, rational functions, etc. or any algebraic expression formed from these functions, whose coefficients are determined by at least one of the following algorithms: linear regression, principal component regression, partial least square regression, ridge regression, lasso or any combination thereof;
any kind of parametric model, such as, but not limited to, polynomial regression models and neural network models;
a nonparametric models, such as, but not limited to, Spline Interpolation, Gaussian Process Models, Multivariate Adaptive Regression Spline, Kernel Regression;
any model, including any one of the explicit possibilities above, built on a previous dimension reduction step, such as, but not limited to, Feature selection, Feature projection, Feature extraction, Principal component analysis (PCA), Non-negative matrix factorization, Kernel PCA, Graph-based kernel PCA, Linear discriminant analysis, Generalized discriminant analysis, Autoencoder, T-distributed Stochastic Neighbor Embedding, Uniform manifold approximation and projection, K-nearest neighbors algorithm, canonical-correlation analysis, low-dimensional embedding, fast approximate K-NN search, locality sensitive hashin, random projection, Multilinear subspace learning, Multilinear principal component analysis, Multilinear independent component analysis, Multilinear linear discriminant analysis, Multilinear canonical correlation analysis, Independent component analysis, Isomap, Kernel PCA, Latent semantic analysis, Partial least squares, Principal component analysis, Multifactor dimensionality reduction, Nonlinear dimensionality reduction, Multilinear Principal Component Analysis, Multilinear subspace learning, Semidefinite embedding, Autoencoder;
any model based on any of the above listed techniques applied on an experimental data set;
any model based on any of the above listed techniques applied on an experimental data set generated by a Design of Experiments approach, such as, but not limited to full factorial designs, fractional factorial designs, D-Optimal designs; or
a model defined by combinations and/or functions and/or chains of the models, functions, and/or algorithms listed above.

Thus, the input unit 12 is, in an example, implemented as an Ethernet interface, a USB™ interface, a wireless interface such as a Wi-Fi™ or Bluetooth™, or 5G or 6G, or any comparable data transfer interface enabling data transfer between input peripherals and the processing unit 14.

The processing unit 14 is configured to (i) perform a multi-objective optimizing process on the system model by exploring a plurality of design configurations by assigning specified values to the set of design parameters, such that the set of primary optimization objective parameters meets a specified system requirement and a design goal over a set of defined constrains, by which the range of at least one of the design parameters is limited.

In an example, the multi-objective optimizing process is a Pareto optimization.

The processing unit 14 is configured to (ii) determine if the multi-objective optimizing process yields a degenerated multi-objective optimal design.

If it is determined that the multi-objective optimizing process yields a degenerated multi-objective optimal design, the processing unit 14 is configured to (iii) perform a further multi-objective optimizing process on the system model using the set of primary optimization objective parameters and at least one secondary optimization objective parameter to provide a multi-objective optimal design.

The secondary objective parameters may include one or more of an optional objective parameter, a objective parameter used in means of a D-Optimal design to explore a maximal variability in the set of design parameters leading to the set of improved primary optimization objective parameters, or an objective parameter including at least one of a determinant, a trace, an eigenvalue, a condition number, or any norm derived from at least one of: a Fisher information Matrix, a transpose of the Fisher Information Matrix, an inverse of the Fisher Information Matrix; or any combinations thereof to be used in means of an optimal design to explore a maximal variability in the set of design parameters leading to the set of improved primary optimization objective parameters. For example, the optimal design may be preferentially in form of: A-Optimality, or C-Optimality, or D-Optimality, or E-Optimality, or T-Optimality, or G-Optimality, or I-Optimality, or V-Optimality. For example, the objective parameter used in means of a D-Optimal design may be the determinant of the Fisher Information Matrix.

Thus, the processing unit 14 may comprise a general-purpose processing unit, a graphics processing unit (GPU), a microcontroller and/or microprocessor, a field programmable gate array (FPGA), a digital signal processor (DSP), and equivalent circuitry, alone or in combination.

Furthermore, such processing unit(s) 14 may be connected to volatile or non-volatile storage, display interfaces, communication interfaces and the like as known to a person skilled in the art.

The output unit 16 is configured to provide the multi-objective optimal design.

Thus, the output unit 16 is, in an example, implemented as an Ethernet interface, a USB™ interface, a wireless interface such as a Wi-Fi™ or Bluetooth™, or 5G or 6G, or any comparable data transfer interface enabling data transfer between output peripherals and the processing unit 14.

In an option, the processing unit 14 is further configured to use an iterative approach to perform Pareto optimization. The processing unit 14 is configured to repeatedly perform the above-described procedures (i) to (iii) until it is determined that the multi-objective optimizing process yields a non-degenerated multi-objective optimal design. The output unit 16 is configured to provide the multi-objective optimal design.

In another option, the processing unit 14 is further configured to use a parallel approach to perform Pareto optimization. The processing unit 14 is configured to provide a user interface allowing a user to interactively navigate along the Pareto frontier based on the set of primary objectives, and to determine a candidate design from designs calculated in response to the interactive navigation that fulfils the optimality conditions of the set of the primary objectives set by the user via the user interface.

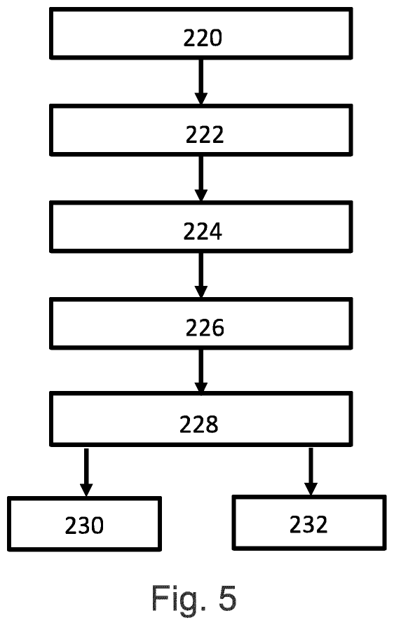

FIG. 5 shows an example of a flowchart for monitoring quality of the chemical mixture in a production process of the chemical mixture having target objective parameters that represent design characteristics of the chemical mixture.

In step 220, the target objective parameters are provided e.g. from a user input. In step 222, the performance characteristic of the produced chemical mixture is provided. The produced chemical mixture has a recipe profile as generated according to the method described therein to meet the target objective parameters.

The performance characteristic may be provided by or derived from measurement data. Such measurement data for instance includes measurement data provided by one or more sensors, such as optical sensors. The one or more sensors may be used to measure the physicochemical properties of the produced chemical mixture. For drug formulation, the measured physicochemical properties could include one or more of the following parameters: solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactibility; hygroscopicity; water content (e.g., loss on drying); concentration of impurities; hardness, chemical resistance, color stain resistance, glass transition temperature; and/or other any other chemical, physicochemical and/or physical properties.

In step 224, the performance characteristic as provided or measured may be compared to the target design characteristics of the chemical mixture to determine if the produced chemical mixture fulfils predetermined quality criteria.

The comparison may performed by comparing one or more physical, chemical or physio-chemical characteristic(s) that relate to the performance characteristic.

Optionally, in step 226, the target design characteristics may be mapped to the performance characteristics. In other word the values corresponding to the performance characteristics may be determined from target design characteristics. In other embodiments the performance characteristic may be mapped to the target design characteristics. Both options are equally applicable.

Optionally, in step 228, the target design characteristics and the performance characteristics or any corresponding values derived therefrom are used for validation. Such validation may be performed by comparing values or value ranges.

If the values lie within an acceptable range or value, such as a 1- or 2-standard deviation(s) interval, the chemical mixture as measured may be valid in the sense that it fulfils the performance criterium or criteria. If the values do not lie within an acceptable range, such as a 1- or 2-standard deviation(s) interval, the chemical mixture as measured may be invalid in the sense that it does not fulfil the performance criterium or criteria.

Optionally, if the chemical mixture is valid, e.g. a control signal for a production process may be triggered in step 230. Such control signal may be associated with the composition of the chemical mixture. It may control dosing equipment for dosing of different components of the chemical mixture in the production process.

Optionally, if the chemical mixture is invalid, e.g. a warning signal for the operator of the production process may be triggered in step 232. Such warning signal may signify the invalidity of the chemical mixture. The invalidity may trigger a stop signal for the production process. In such cases, the recipe profile may be updated for the production of the chemical mixture to achieve the target design characteristics of the chemical mixture.

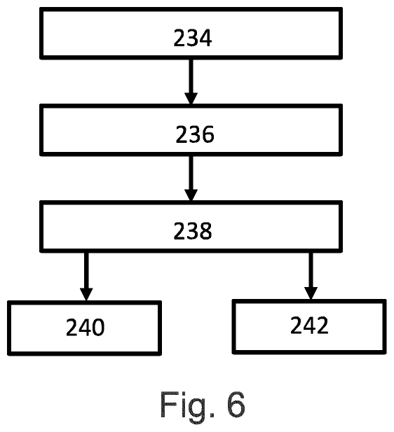

FIG. 6 shows an example of a flowchart for validating the production of the chemical mixture.

In step 234, an existing performance characteristic (e.g. one or more measured physicochemical properties) for a chemical mixture is provided, which has been produced from validated precursors.

In step 236, based on the existing performance characteristic a recipe profile is generated according to the method described therein that includes an ingredient identifier and related property data, which are associated with at least one new precursor.

In step 238, the performance characteristic of a chemical mixture produced based on the recipe profile and the existing performance characteristics are compared to validate the at least one new precursor. If the comparison lies within an acceptable range, the at least one new precursor is valid. On the other hand, if the comparison does not lie within the acceptable range, the at least one new precursor is invalid.

If new precursor(s) is valid, e.g. control signal is generated for a production process based on the new precursor(s) may be triggered in step 240. Such control signal may by be associated with the composition of the chemical mixture including the new precursor. It may control dosing equipment configured to dose different components of the chemical mixture in the production process.

If the chemical mixture is invalid, e.g. a warning signal for the operator of the production process may be triggered in step 242. Such warning signal may signify the invalidity of the new precursor(s). This may trigger a stop signal for the production process.

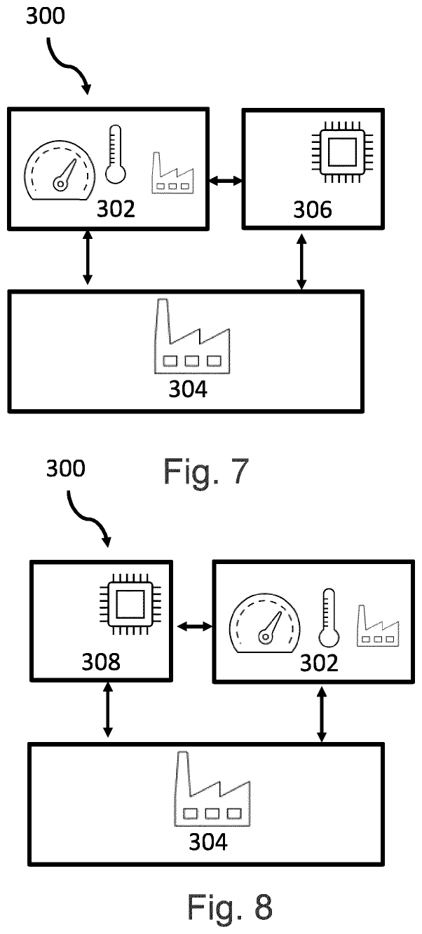

FIG. 7 shows an example of a production line 300 for producing the chemical mixture with a monitoring apparatus 306.

The production line 300 may include dosing equipment 302 configured to dose different precursors of the chemical mixture in the production process. The production line may include a conveyor system 304 to convey e.g. bottles, plastic packaging or other suitable packaging to be filled with the chemical mixture. The production line may include a monitoring apparatus 306 configured to monitor quality of the chemical mixture in a production process of the chemical mixture.

The monitoring apparatus 306 and/or the dosing equipment apparatus 302 may be configured to receive a target design characteristics of the chemical mixture. The target design characteristics may specify the composition data for the chemical mixture including one or more ingredients. The target design characteristics may include quality criteria like physicochemical properties. The monitoring apparatus may be configured to provide the composition data to the dosing equipment and vice versa. The dosing equipment may be configured to control the dosing based on the provided composition data.

The monitoring apparatus 306 may be configured to measure one or more performance characteristic(s). The monitoring apparatus 306 may be configured to compare the physicochemical properties, or any value derived from the physicochemical properties to the measured performance characteristic(s). If the comparison lies within an acceptable range or value, the produced chemical mixture fulfills quality criteria. If the comparison does not lie within an acceptable range or value, the produced chemical mixture does not fulfill quality criteria. In the latter case the monitoring unit may be configured to notify an operator or to provide adjusted composition data to the dosing equipment 302.

FIG. 8 shows another example of a production line 300 for producing the chemical mixture with a validation apparatus 308.

The production line 300 may include dosing equipment 302 configured to dose different precursors of the chemical mixture in the production process. The production line 300 may include a conveyor system 304 to convey e.g. bottles, plastic packaging or other suitable packaging to be filled with the chemical mixture. The production line 300 may include a validation apparatus 308 configured to validate the production of the chemical mixture.

The validation apparatus 308 may be configured to receive an existing performance characteristic of the chemical mixture (e.g. two or more physicochemical properties or any value derived from the physicochemical properties). The validation apparatus 308 may be configured to generate a recipe profile based on the existing performance characteristic. The recipe profile may comprise new precursor(s). The validation apparatus 308 may be configured to receive one or more data associated with the new precursor(s). The validation apparatus 308 may be configured to validate the new precursor(s) for production of the chemical mixture. The validation apparatus 308 may be configured to compare a performance characteristic of a chemical mixture produced using the new recipe profile and the existing performance characteristic. This way not only the production of the chemical mixture but also its application may be validated. The validation apparatus 308 may be configured to provide the composition data including the new precursor(s) to the dosing equipment and vice versa.

Combinations and modifications of the embodiments shown in FIGS. 5 and 6 are similarly possible. Both methods exemplify the strength of the methods described herein. This allows for simplified and more reliable production through monitoring production of the chemical mixture or through validating new precursor(s) to be used for producing the chemical mixture.

All definitions, as defined and used herein, should be understood to control over dictionary definitions, definitions in documents incorporated by reference, and/or ordinary meanings of the defined terms.

The indefinite articles “a” and “an”, as used herein in the specification and in the claims, unless clearly indicated to the contrary, should be understood to mean “at least one”.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either”, “one of”, “only one of”, or “exactly one of”.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising”, “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Furthermore, in this detailed description, a person skilled in the art should note that quantitative qualifying terms such as “generally”, “substantially”, “mostly”, and other terms are used, in general, to mean that the referred to object, characteristic, or quality constitutes a majority of the subject of the reference. The meaning of any of these terms is dependent upon the context within which it is used, and the meaning may be expressly modified.

In another exemplary embodiment of the present invention, a computer program or a computer program element is provided that is characterized by being adapted to execute the method steps of the method according to one of the preceding embodiments, on an appropriate system. The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up date turns an existing program into a program that uses the invention.

Further on, the computer program element might be able to provide all necessary steps to fulfil the procedure of an exemplary embodiment of the method as described above. According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. The computer program may also be distributed by printing the source code in a book, e.g. “Numerical Recipes”. According to a further exemplary embodiment of the present invention, a medium for making a computer program element available for downloading is provided, which computer program element is arranged to perform a method according to one of the previously described embodiments of the invention.

All features can be combined to provide a synergetic effect that is more than the simple summation of the features.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A computer-implemented method for generating a recipe profile of a chemical mixture, the method comprising:
a) providing, via an input channel, a system model for modelling the chemical mixture, which associates a set of design parameters with a plurality of objective parameters that represent design characteristics of the chemical mixture, wherein the set of design parameters comprises a chemical mixture recipe having two or more ingredients, and the plurality of objective parameters comprises two or more physicochemical properties of the chemical mixture;
b) defining, via the input channel, a set of primary optimization objective parameters, wherein the set of primary optimization objective parameters comprises one or more essential physicochemical properties of the chemical mixture;
c) performing, by a processor, a multi-objective optimizing process on the system model by exploring a plurality of design configurations by assigning specified values to the set of design parameters, such that the set of primary optimization objective parameters meets a specified system requirement and a design goal over a set of defined constrains, by which the range of at least one of the design parameters is limited;
d) determining, by the processor, if the multi-objective optimizing process yields a degenerated multi-objective optimal design;
e) if it is determined that the multi-objective optimizing process yields a degenerated multi-objective optimal design, performing, by the processor, a further multi-objective optimizing process on the system model using the set of primary optimization objective parameters and at least one secondary optimization objective parameter to provide a multi-objective optimal design, wherein the at least one secondary optimization objective parameter comprises one or more optional physicochemical properties of the chemical mixture; and
f) providing, via an output channel, the multi-objective optimal design that comprises a recipe profile preferably usable for production of the chemical mixture.

2. The computer implemented method according to claim 1, wherein the set of design parameters further comprises a process condition for producing the chemical mixture.

3. The computer implemented method according to claim 1, further comprising:
repeatedly performing steps c) to e), until it is determined that the multi-objective optimizing process yields a non-degenerated multi-objective optimal design; and
providing, via the output channel, the non-degenerated multi-objective optimal design.

4. The computer implemented method according to claim 1,
wherein step e) further comprises:
performing a further multi-objective optimizing process on the system model using the set of primary optimization objective parameters and a set of multiple secondary objectives defining a Pareto optimization task, for which a Pareto frontier is computed and stored;
providing a user interface allowing a user to interactively navigate along the Pareto frontier based on the set of multiple secondary objectives; and
determining a candidate design from designs calculated in response to the interactive navigation that fulfils the optimality conditions of the set of multiple secondary objectives set by the user via the user interface.

5. The computer-implemented method according to claim 1,
wherein step c) further comprises:
providing a user interface allowing a user to interactively navigate along the Pareto frontier based on the set of primary objectives; and
determining a candidate design from designs calculated in response to the interactive navigation that fulfils the optimality conditions of the set of the primary objectives set by the user via the user interface.

6. The computer implemented method according to claim 1,
wherein the chemical mixture comprises one or more of: paint formulation, agricultural multi-component mixture, pharmaceutical multi-component mixture, nutrition multi-component mixture, ink multi-component mixture, chemical mixture for construction purposes, and chemical mixture used inside oil production.

7. A method for monitoring production of a chemical mixture, the method comprising:
providing a plurality of target objective parameters that represent design characteristics of the chemical mixture;
providing a performance characteristic of a produced chemical mixture that has a recipe profile generated according to the method of any one of the preceding claims; and
comparing the performance characteristic with the design characteristics of the chemical mixture to determine if the produced chemical mixture fulfils predetermined quality criteria.

8. The method for validating production of a chemical mixture, the method comprising:
providing an existing performance characteristic for a chemical mixture that has been produced from validated precursors;
generating a recipe profile based on the existing performance characteristic according to the method of claim 1, wherein the recipe profile comprises an ingredient identifier and related property data, which are associated with at least one new precursor; and
comparing a performance characteristic of a chemical mixture produced using the recipe profile and the existing performance characteristic to validate the at least one new precursor.

9. An apparatus for generating a recipe profile of a chemical mixture, the apparatus comprising one or more processing unit(s) configured to generate the recipe profile of the chemical mixture, wherein the processing unit(s) include instructions, which when executed on the one or more processing unit(s) execute the method claim 1.

10. An apparatus for monitoring production of a chemical mixture, the apparatus comprising one or more processing unit(s) configured to monitor production, wherein the processing unit(s) include instructions, which when executed on the one or more processing unit(s) execute the method claim 7.

11. An apparatus for validating production of a chemical mixture, the apparatus comprising one or more processing unit(s) configured to validate production, wherein the processing unit(s) include instructions, which when executed on the one or more processing unit(s) execute the method claim 8.

12. A computer program element comprising instructions, which when executed by a processing unit, cause the processing unit to carry out the steps of the method of claim 1.

13. A computer-readable medium having stored the program element of claim 12.

14. (canceled)
